(19) United States
(12) Patent Application Publication
(10) Pub. No.: US 2025/0302839 A1
(43) Pub. Date: Oct. 2, 2025

(54) COMPOSITIONS AND METHODS FOR DELIVERY OF RIBOFLAVIN

(71) Applicant: D&D Biopharmaceuticals, Inc., Sterling, VA (US)

(72) Inventors: Bruce H. DeWOOLFSON, Sterling, VA (US); Dale P. DeVORE, Chelmsford, MA (US); Debra SKELNIK, Cambridge, MN (US)

(73) Assignee: D&D Biopharmaceuticals, Inc., Sterling, VA (US)

(21) Appl. No.: 18/623,345

(22) Filed: Apr. 1, 2024

(51) Int. Cl. A61K 31/525; A61K 9/00; A61K 41/00; A61K 47/02; A61K 47/12; A61K 47/20; A61N 5/06

(52) U.S. Cl. CPC A61K 31/525; A61K 9/0048; A61K 41/0057; A61K 47/02; A61K 47/12; A61K 47/20; A61N 5/062; A61N 2005/0661

(57) ABSTRACT

Methods of treating eyes are described. The method may include administering an agent capable of disrupting corneal cell junctures to a corneal surface of the eye, the corneal surface comprising an intact epithelium and stroma; and administering riboflavin to the corneal surface after or simultaneously with the agent. The riboflavin may diffuse through the eye to penetrate the stroma within 10 minutes of administration of the riboflavin.

### TECHNICAL FIELD

The present disclosure relates to methods of topical delivery of riboflavin to the cornea. The methods herein may facilitate various ocular procedures, such as treating keratoconus.

### BACKGROUND

Administering drugs to the eye presents various challenges. While topical formulations such as eye drops are a convenient route of administration, it can be difficult to ensure that a drug penetrates the cornea to reach the intended target site. This is especially challenging for active agents that are large biomolecules. Additionally, due to the protective structure of the cornea, repeated administration of drops over several hours is often necessary to deliver sufficient active agent to the interior of the eye.

The human cornea includes three primary layers: epithelium, stroma, and endothelium. The thickness of the cornea is normally 500-600 μm, about 90% being stroma. The epithelium is approximately 50 μm thick and contains 5-6 layers of cells with tight junctures between the cells, especially the first 2 layers of flattened, plate-like superficial cells. The next 2-3 layers contain wing-like or polygonal cells over a single row of columnar basal cells. The epithelium forms an effective permeability barrier especially to polar and ionic molecules. Conversely, lipophilic molecules may be absorbed across the epithelium. Molecular size effects penetration of ionic and hydrophilic molecules. Permeability of such molecules is generally limited to a molecular size of 500 daltons (Da). The next barrier below the epithelium is Bowman's Membrane. Bowman's Membrane is an 8-14 μm thick homogenous sheet separating the epithelium from the underlying, acellular stroma (substantia propria).

The stroma includes 200-250 alternating lamellae (layers) of collagen fibers. Each lamellae is about 1 μm thick and 10-25 μm wide. The stroma contains about 70% water and impedes movement of molecules greater than 500 Da. Collagen fibers make up a majority of the structure of the cornea. Proteoglycans and fiber associated collagens are linked to collagen fibers to control diameter and stabilize stromal architecture. Fiber associated proteoglycans include a category called small leucine-rich proteoglycans and includes decorin, biglycan, keratocan, lumican, mimican, and fibromodulin. Fiber associated collagens encompass a category known as fibril associated collagen molecules with interrupted triple helices (FACITs) and includes Type VI, Type X, Type XII, and Type XIV collagen.

Keratoconus is a degeneration disorder of the eye in which structural changes in the cornea cause it to thin and change to a more conical shape than the normal gradual curve (cone). The incidence and prevalence of keratoconus in the U.S. population is generally estimated at about 55 diagnosed individuals per 100,000 or approximately 1 in 2,000 individuals. Some estimates suggest the incidence may be as high as 1 in 400 individuals. In addition 20% of keratoconic patients may suffer severe visual deterioration due to irregular astigmatism, myopia, corneal scarring and optical means such as spectacles and rigid gas permeable contact lenses do not offer any visual rehabilitation.

A significant challenge in the treatment of keratoconus and other ocular conditions is administering active agents, often large biomolecules. The primary permeability barrier of the cornea to hydrophilic molecules resides largely within the superficial, top two layers, of the epithelium. This is due to the annular tight junctures which surround the superficial epithelial cells sealing the epithelium to all but very small hydrophilic molecules, generally less than 500 Da in size. Physical methods to open epithelial channels have included application of ocular retention rings, D-alpha-tocopheryl (polyethylene glycol) 1000 VE-TPGS treatment as a penetration enhancer, and producing intrastromal channels by injection. Other methods have included application of drug delivery devices, delivering drug eluting elements to the eye surface, and application of microneedles.

Prior methods of treating keratoconus typically take more than one hour. In one such procedure, for example, the epithelium is removed by mechanical debridement, riboflavin applied by drops every 5 minutes for 30 minutes, and then the eye irradiated for 30 minutes during which additional riboflavin drops may be applied. In another procedure, the epithelium is kept intact requiring repeated, periodic administration application of riboflavin followed by exposure to UV irradiation, again providing a treatment over an hour. Such considerable treatment times are often inconvenient and uncomfortable for patients, often resulting in prolonged pain, delayed healing, infection, scarring, and slow visual recovery.

### SUMMARY

The present disclosure includes methods of treating an eye or both eyes of a subject, e.g., a patient, by administering an agent capable of disrupting corneal cell junctures to a corneal surface of the eye, the corneal surface comprising an intact epithelium and stroma; and administering a therapeutically-effective amount of riboflavin to the corneal surface after or simultaneously with the agent. The agent may comprise, for example, an anhydride, an acid chloride, a sulfonyl chloride, or a sulfonic acid. In at least one example, the agent comprises glutaric anhydride. The riboflavin may diffuse through the eye(s) to penetrate the stroma, e.g., within 10 minutes after administering the riboflavin. Administering the agent to the corneal surface may include contacting the corneal surface with a buffered solution comprising the agent for at least 30 seconds. In some examples, the solution has a pH of from 7.5 to 9.0. The solution may comprise, for example, dibasic sodium phosphate, monobasic sodium phosphate, disodium phosphate, or a mixture thereof. Further, for example, the solution may have a concentration of the agent ranging from about 1.0 mg/mL to about 5.0 mg/mL. The agent may dissociate molecular bridges between stromal collagen fibers. The solution may be administered using an applicator placed against the corneal surface.

In some examples, the solution comprising the agent also comprises the therapeutically-effective amount of riboflavin. Alternatively, the solution comprising the agent does not comprise the therapeutically-effective amount of riboflavin, e.g., the solution comprising riboflavin being a different solution. In some examples, the therapeutically-effective amount of riboflavin may be administered within 5 minutes after administering the agent. The therapeutically-effective amount of riboflavin may administered as a solution having a concentration of about 0.5 mg/mL to about 5.0 mg/mL riboflavin, the solution comprising riboflavin being the same or different than the solution comprising the agent. The therapeutically-effective amount of riboflavin may be administered to the corneal surface by a plurality of drops, e.g., continuous drops, and/or using an applicator placed against the corneal surface. In at least one example, the therapeutically-effective amount of riboflavin corresponds to a total volume of riboflavin in solution ranging from about 0.1 mL to about 1.0 mL. According to some aspects, at least 50%, at least 75%, or at least 90% of the therapeutically-effective amount of riboflavin administered to the corneal surface may diffuse into the stroma within 5 minutes of administering the riboflavin. Additionally or alternatively, at least 75% of the therapeutically-effective amount of riboflavin administered to the corneal surface may diffuse into the stroma within 2 minutes of administering the therapeutically-effective amount of riboflavin.

According to some aspects herein, the method further comprises administering a buffer solution or physiological saline solution to the corneal surface before administering the agent, e.g., to deprotonate free amines on corneal proteins. The buffer solution or physiological saline solution may have a concentration ranging from 0.05 M to 1.0 M, for example. Additionally or alternatively, the method may further comprise irradiating the eye with UVA light. The methods herein may be conducted under hyperoxic conditions.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate various exemplary embodiments and together with the description, serve to explain the principles of the disclosed embodiments.

FIGS. 1A-1C show pre-treatment evaluation of test and control human corneas, discussed in Example 1.

FIGS. 2A-2C show post-treatment evaluation of test and control human corneas, discussed in Example 1.

FIG. 3 shows riboflavin fluorescence of a test cornea sample, discussed in Example 1.

FIG. 4 shows riboflavin fluorescence of a control cornea sample, discussed in Example 1.

FIG. 5 shows a comparison of riboflavin fluorescence of a test sample (top panel) and control sample (bottom panel), discussed in Example 1.

### DETAILED DESCRIPTION

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass ±5% of a specified amount or value.

The present disclosure describes methods of treating the cornea by topical delivery of riboflavin to the stroma, after or in conjunction with applying an agent capable of disrupting cell junctures to allow diffusion of the riboflavin into the stroma. The riboflavin may diffuse through the eye to penetrate the stroma, Descemet's membrane, and the endothelium.

Riboflavin has a molecular weight 376.4 Da (456.3 Da for riboflavin 5-phosphate) and due to its relatively large size, is unable to pass through the untreated cornea. Riboflavin is a B vitamin involved in a wide variety of cellular processes. The vitamin riboflavin is distributed in tissues at relatively small concentrations. Riboflavin is readily absorbed from the gastrointestinal tract with excretion correlated with the amounts ingested. This vitamin plays an important role in metabolism of ketone bodies, carbohydrates, proteins and fats, as well other aspects of energy metabolism. Riboflavin may serve as a photosensitizer of photochemical cross-linking reactions and promote the production of reactive singlet-oxygen; and may act as an UV absorber to protect the endothelium, lens, and retina from excessive UV irradiation.

According to the methods herein, riboflavin may penetrate the epithelium and enter corneal stroma. The riboflavin thus delivered may permit stromal stabilization when exposed to UVA irradiation. Stabilization of the corneal curvature resulting from orthokeratology procedures may provide a long-term, non-invasive treatment for myopia, hyperopia and astigmatism. The riboflavin may comprise riboflavin 5-phosphate, e.g., riboflavin 5-phosphate sodium in dextran solution. Exemplary riboflavin solutions that may be used according to the present disclosure include, but are not limited to, Photrexa®, Photrexa Viscous®, Safecross®, VibeX Xtra™, VibeX Rapid™, ParaCel™, and MedioCROSS® formulations.

In an exemplary method, an agent such as an acylation agent or acetylation agent is applied to the cornea to disrupt tight corneal junctures, followed by or with simultaneous application of riboflavin. After riboflavin is applied, UVA irradiation may be performed for treating keratoconus. Without being bound by theory, it is believed that UVA irradiation of riboflavin in the stroma creates reactive species that promote formation of new bonds to strengthen collagen fibers in the corneal stroma while protecting the lens and retina from UV damage. For example, according to some aspects of the present disclosure, UVA irradiation may be performed with 370 nm light for about 5 minutes to about 30 minutes. Additional steps may include applying an anesthetic, and/or applying a buffer before and/or after the agent.

Agents suitable for the present disclosure include, but are not limited to, anhydrides, acid chlorides, sulfonyl chlorides, and sulfonic acids. The type of acylation agent that results in cell juncture disruption may be different from the type of acylation agent that results in dissociation of molecular bridges between stromal collagen fibers, without producing corneal swelling. The latter includes agents that substitute a non-charged moiety or (a positively charged moiety) to a deprotonated amine. Substitution with a negatively charged moiety has been shown to result in “hardening” of the treated tissue.

Suitable anhydrides include agents that change the net charge from positive to negative. These agents include, but are not limited to, anhydrides including maleic anhydride, succinic anhydride, glutaric anhydride, citractonic anhydride, methyl succinic anhydride, itaconic anhydride, methyl dimethyl glutaric anhydride, acetic anhydride, propionic anhydride, methacrylic anhydride, butyric anhydride, isobutryic anhydride, valeic anhydride, hexanoic anhydride, decanoic anhydride, dodecanoic anhydride, myristic anhydride, palmitic anhydride, oleic anhydride, and phthalic anhydride, among others. Exemplary acid chlorides include, but are not limited to, oxalyl chloride, propionyl chloride, methacryloyl chloride, acryloyl chloride, methacryloyl chloride, butyryl chloride, isobutyryl chloride, valeryl chloride, isovaleryl chloride, hexanoly chloride, heptanoly chloride, and malonyl chloride. Exemplary sulfonyl chlorides include, but are not limited to, chlorosulfonylacetyl chloride, chlorosulfonylbenzoic acid, 4-chloro-3-(chlorosulfonyl)-5-nitroebnzoic acid, 1-hexadecanesulfonyl chloride, 4-(hexadecyloxy)benzenesulfonyl chloride, pentamethylbenzenesulfonyl chloride, 4-tert-butylbenzenesulfonyl chloride, tolulenesulfonyl chloride, 2,5 dimethylbenzenesulfonyl chloride, and 3-(chlorosulfonyl)-P-anisic acid. Exemplary sulfonyl acids include, but are not limited to, 5-tridecyl-1-2, oxathiolane-2,2-dioxide. Exemplary sulfonic acids include, but are not limited to, 3-sulfobenzoic acid. Other agents suitable for the methods herein can change the net charge from one positive to two negatives per reacted site. Such agents include, but are not limited to, 3,5-dicarboxy-benzenesulfonyl chloride. Still other agents can be used to change the net charge from positive to neutral per reacted site. Such agents include, but are not limited to, anhydrides including acetic anhydride, chloroacetic anhydride, propionic anhydride, butyric anhydride, isobutyric anhydride, isovaleric anhydride, hexanoic anhydride, and other anhydrides; acid chlorides including acetyl chloride, propionyl chloride, dichloropropionyl chloride, butyryl chloride, isobutyryl chloride, valeryl chloride, and others; sulfonyl chlorides including, but not limited to, ethane sulfonyl chloride, methane sulfonyl chloride, 1-butane sulfonyl chloride and others. In some examples herein, the agent comprises glutaric anhydride, butyric anhydride, or propionic anhydride. Without being bound by theory, it is believed that the agents may be capable of dissociating molecular bridges between cells and between stromal collagen fibers without causing corneal swelling.

The agent may be diluted in a physiologically acceptable solution at alkaline pH, e.g., slightly alkaline pH. For example, the solution may have a pH between 7.5 and 9, such as between 7.5 and 8.5, or between 8 and 8.8, e.g., a pH between 8.3 and 8.8. In at least one example, the solution used to dilute the agent may comprise a buffer, such as disodium phosphate solution. The concentration of agent may range from about 1.0 mg/ml to about 30.0 mg/mL, from about 1.0 mg/mL to about 20.0 mg/mL, from about 1.0 mg/ml to about 10.0 mg/mL, such as from about 1.0 mg/mL to about 5.0 mg/mL, from about 3.0 mg/mL to about 5.0 mg/mL, from about 5.0 mg/mL to about 10.0 mg/mL, from about 4.0 mg/mL to about 5.0 mg/mL, from about 10.0 mg/mL to about 15.0 mg/mL, from about 15 mg/mL to about 30 mg/mL, or from about 8.0 mg/mL to about 12.0 mg/mL.

The agent solution may be applied directly to the corneal surface, e.g., using an applicator placed on the corneal surface or by continuous drops. The agent solution may be applied to the corneal surface after first priming the corneal tissue with the slightly alkaline pH solution or buffer, e.g., a pretreatment buffer. Agents such as acylation agents generally react with proteins that have first been deprotonated, or hydrolyze into acids. After applying the agent, the buffer may be applied to the eye.

In at least one example, the agent comprises glutaric anhydride. Glutaric anhydride is a powder and may be rapidly dissolved in a buffer, e.g., a pretreatment buffer, before administration to the cornea. The glutaric anhydride powder may be pulverized using a mortar and pestle or otherwise ground to reduce the particle size to allow rapid dissolution.

As mentioned above, the agent capable of disrupting cell junctures may be applied to the cornea before and/or simultaneously with riboflavin. For example, a composition suitable for direct application to the cornea may comprise glutaric anhydride, a physiologically acceptable solution at alkaline pH (e.g., sodium phosphate buffer), and riboflavin. The ability to deliver riboflavin, a relatively large biomolecule, without removal of the epithelium, in a single step, may facilitate treatment of keratoconus and greatly reduce the time involved in such treatment.

The present disclosure includes methods for treating corneas comprising administering to the corneal surface a therapeutically effective amount of an agent to permit diffusion of riboflavin to the stroma. Without being bound by theory, it is believed that the methods herein may be capable of dissociating ionically bound bridging molecules from stromal collagen fibers to temporarily destabilize such stromal collagen network, e.g., such that the network can be restabilized in the desired configuration to treat keratoconus with UVA irradiation. An exemplary non-limiting concentration of riboflavin used in some examples herein is about 0.5 mg/mL to about 5.0 mg/mL, such as from about 1.2 mg/mL to about 3.0 mg/mL. Optionally, the riboflavin may be provided simultaneously with a physiologically acceptable solution that comprises the agent(s) that permit diffusion of riboflavin to the stroma.

In accordance with the present disclosure, there is provided a method for treating the cornea with one or more agents to permit penetration of riboflavin to treat an ocular condition and/or as part of a subsequent procedure to treat an ocular condition. For example, the methods herein may include subsequent exposure to UVA irradiation to increase biomechanical stability of corneal stroma for treatment of keratonus. Optionally, the methods herein may be performed under hyperoxic conditions, e.g., to facilitate efficient corneal crosslinking.

The agent(s) and riboflavin may be applied simultaneously or sequentially to the cornea by direct administration using an applicator, e.g., the applicator being in contact with the corneal surface. Exemplary applicators are described in U.S. Pat. No. 11,259,959 and U.S. application Ser. No. 18/072,163 filed on Nov. 30, 2022. The agent and/or riboflavin may be dissolved or diluted in a physiologically acceptable solution (the same solution or separate solutions) shortly or immediately prior to treatment and placed into the applicator. The solution(s) then may be delivered via the same applicator or separate applicators (in contact with the corneal surface) to thereby expose the surface of the cornea to the agent(s) and riboflavin for a suitable amount of time. For example, the cornea may be exposed to the agent(s) and riboflavin simultaneously or in sequential administration for about 2 seconds to about 1 minute or 2 minutes, such as from about 15 seconds to about 45 seconds, from about 30 seconds to about 60 seconds, about 25 seconds to about 35 seconds, or about 1 minute to about 2 minutes. Additionally or alternatively, the agent(s) and/or riboflavin may be administered to the corneal surface dropwise, e.g., by continuous drops. In some examples herein, a single administration of the agent(s) and riboflavin solution(s) may be sufficient to diffuse into the stroma. For example, a single administration of several drops of the riboflavin in solution may be sufficient to saturate the stroma within 10 minutes or within 5 minutes, e.g., in less than or equal to 2 minutes, or less than or equal to 1 minute. In some aspects, the estimated average endothelial riboflavin concentration may be greater than about 0.01% wt., such as ranging from about 0.01% wt. to about 0.1% wt.

According to some aspects of the present disclosure, the amount of agent administered to the corneal surface may be equal to or greater than the amount of riboflavin administered to the corneal surface. For example, the weight ratio of agent to riboflavin that is administered may range from about 1:1 to about 10:1, or from about 1:1 to about 5:1, or from about 1:1 to about 2:1. In other aspects, the amount of agent administered to the corneal surface may be less than the amount of riboflavin administered to the corneal surface. For example, the weight ratio of agent to riboflavin that is administered may be less than 1, such as ranging from about 1:5 to less than 1:1, from about 1:5 to about 1:2, or from about 1:3 to about 1:2.

In some examples, the total volume of riboflavin solution administered to the corneal surface may range from about 0.1 mL to about 1.0 mL, such as from about 0.2 mL to about 0.8 mL, from about 0.5 mL to about 1.0 mL, from about 0.4 mL to about 0.7 mL, e.g., the riboflavin solution having a concentration ranging from about 1.0 mg/ml to about 10.0 mg/mL. The total amount of riboflavin administered (e.g., in solution) may be selected to be sufficient to saturate the stroma when the riboflavin diffuses through the corneal to reach the stroma, e.g., a therapeutically-effective amount of riboflavin. According to some aspects, a therapeutically-effective amount of riboflavin administered to the corneal surface in solution may range from about 0.5 mL to about 5.0 mL, having a concentration of about 1.0 mg/mL to about 5.0 mg/mL. The therapeutically-effective amount may be sufficient to saturate the stroma. In some aspects, the administration of riboflavin may be repeated one or more times to provide the therapeutically-effective amount.

An exemplary method according to the present disclosure may include the following steps (optionally under hyperoxic conditions):
1. Apply drops of topical anesthetic; wait for about 2 minutes;
2. Apply pretreatment buffer (e.g., sodium phosphate buffer solution, pH of about 8.4-8.5) to the eye for about 30-60 seconds;
3. Apply acylation agent in pretreatment buffer to the eye for about 30 seconds to about 2 minutes;
4. Rinse the eye thoroughly with sterile buffer solution or sterile physiological saline solution;
5. Apply solution of riboflavin for about 30 seconds; and
6. Rinse the eye thoroughly with sterile buffer solution or sterile physiological saline solution.

Another exemplary method according to the present disclosure may include the following steps (optionally under hyperoxic conditions):
1. Apply drops of topical anesthetic; wait for about 2 minutes;
2. Apply pretreatment buffer (e.g., sodium phosphate buffer solution, pH of about 8.4-8.5) to the eye for about 30 seconds;
3. Apply composition that includes acylation agent and riboflavin in pretreatment buffer to the eye for about 30 seconds to 2 minutes; and
4. Rinse the eye thoroughly with sterile buffer solution or sterile physiological saline solution.

The methods herein may permit diffusion of riboflavin into the stromal matrix, without removing the epithelium, in a time of less than 30 minutes, such as less than 20 minutes, less than 15 minutes, less than 10 minutes, less than 7 minutes, less than 5 minutes, less than 3 minutes, or less than 2 minutes, such as a time of 30 seconds to 10 minutes, 1-5 minutes, 2-7 minutes, 2-5 minutes, or 1-3 minutes. At least 50% of the riboflavin administered in a single application (such as, e.g., 1 mL of a riboflavin solution having a concentration of about 1.0 mg/mL to about 10.0 mg/mL) may diffuse into the stroma, e.g., at least 60%, at least 70%, at least 80%, at least 90%, or at least 95%. For example, the stroma may be saturated with the riboflavin in a time of less than 10 minutes, e.g., in a time of 1-5 minutes. In at least one example, the stroma may be saturated with the riboflavin in a time of less than 2 minutes, e.g., in a time of 30 seconds to less than 2 minutes.

Another exemplary procedure according to the present disclosure includes the following steps (optionally under hyperoxic conditions):
1. Apply several drops of topical anesthetic; wait for about 2 minutes;
2. Apply pretreatment buffer (e.g., sodium phosphate buffer solution, pH of about 8.4-8.5) to the eye for about 30 seconds;
3. Apply buffered glutaric anhydride solution (concentration 8-12 mg/mL) to the corneal surface for about 30-60 seconds;
4. Rinse the eye thoroughly with sterile buffer solution or sterile physiological saline solution;
5. Apply 1.0 mL riboflavin 5-phosphate having a concentration of 1-2 mg/ml for about 30 seconds to 2 minutes; and
6. Rinse the eye thoroughly with sterile buffer solution or sterile physiological saline solution.

Another exemplary procedure according to the present disclosure includes the following steps (optionally under hyperoxic conditions):
1. Apply several drops of topical anesthetic; wait for about 2 minutes;
2. Apply pretreatment buffer (e.g., sodium phosphate buffer solution, pH of about 8.4-8.5) to the eye for about 30 seconds;
3. Apply composition that in includes buffered glutaric anhydride solution (concentration 8-12 mg/mL) and riboflavin 5-phosphate (concentration of 1-2 mg/mL) to the corneal surface for about 30 seconds to 2 minutes; and
4. Rinse the eye thoroughly with sterile buffer solution or sterile physiological saline solution.

It can therefore be seen that the present disclosure provides unique and effective methods to permit topical delivery and diffusion of riboflavin through the corneal surface into the stromal matrix.

The following examples are intended to illustrate the present disclosure without, however, being limiting in nature. It is understood that the present disclosure encompasses additional embodiments consistent with the foregoing description and following examples.

### EXAMPLES

### Example 1

The objective of this study was to evaluate penetration of riboflavin into a human donor cornea sample after treatment with an agent to disrupt corneal junctures, as compared to a control without treatment by the agent. The agent used in this example was glutaric anhydride (GA). Paired human donor corneas were evaluated prior to treatment and immediately after treatment for intact epithelium, stroma, and endothelium. Riboflavin fluorescence for the GA treatment and the non-GA treatment control were compared. The test human cornea sample was labeled 21-095656 OD, and the control human cornea sample was labeled 21-095656 OS.

### Procurement of Donor Cornea

One pair human corneas were recovered by in situ removal by a certified eyebank technician and placed in LIFE4° C. and stored in Bausch & Lomb 20 ml viewing chambers, and stored at 2-8° C. Corneas were shipped on wet ice overnight to the treatment facility. Upon arrival, the corneas were warmed for 2±2 hours at 35° C. and evaluated for corneal integrity. Corneas were stored at 2-8° C. for a total of 14 days from the time of death in Life4° C. corneal preservation medium in Krolman 20 ml viewing chambers. At the time of the study, corneas were warmed for 2±2 hours at 35° C., and evaluated for corneal integrity.

### Treatment Procedure for Control and Treated Human Cornea Samples

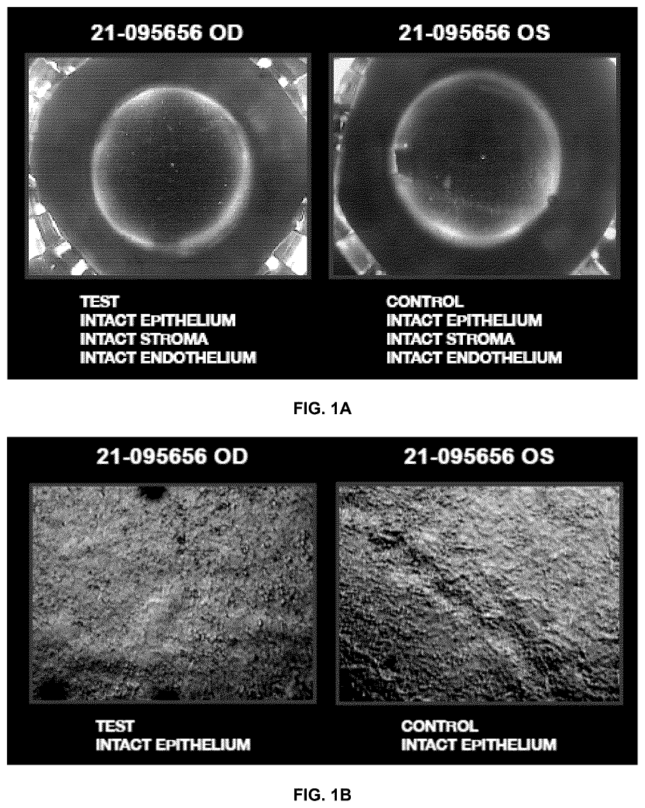

The control and treated cornea samples were placed in a petri dish with the epithelial side up in a laminar flow hood with enough Life4° C. corneal preservation medium to fill the endothelial side of the human cornea with medium. This allowed exposure to the corneal surface in a fixed position with protection of the endothelial surface. Each sample was analyzed before treatment (FIGS. 1A-1C).

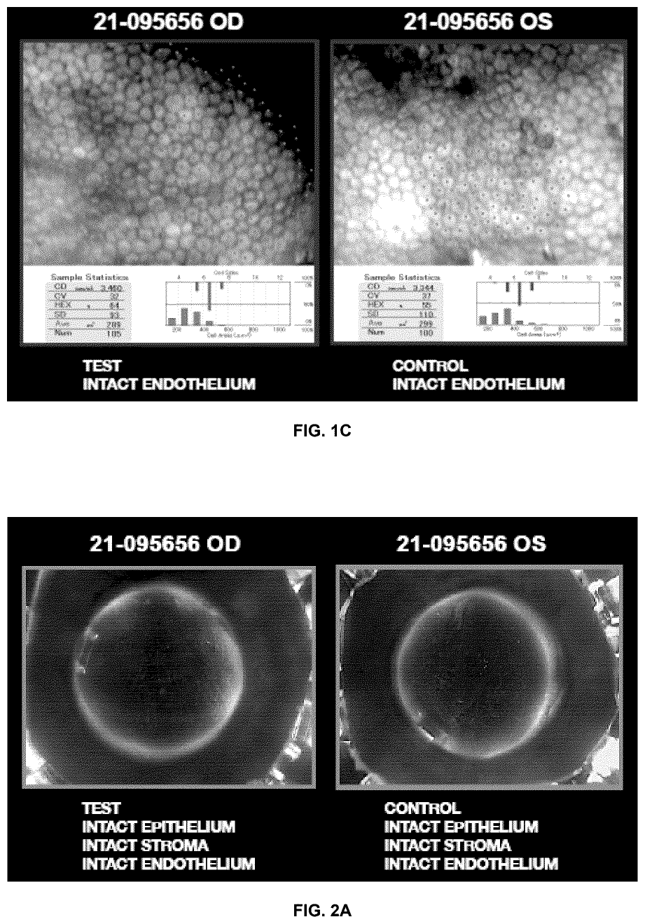

The control cornea was treated with 1.0 ml of 0.1M sodium phosphate buffer, (pH 7.2) for 1 minute, with continuous drops; further treated with 0.5 mL of 0.1M sodium phosphate buffer, (pH 8.5) for 30 seconds with continuous drops; and further treated with 0.5 mL of 0.1M sodium phosphate buffer, (pH 7.2) for 30 seconds, with continuous drops and then further treated with 0.5 mL of riboflavin (1.46 mg/ml in PBS) for 60 seconds, with continuous drops; again further treated with 0.5 mL of riboflavin (1.46 mg/ml in PBS) for 60 seconds, with continuous drops; rinsed with 1.0 ml of 0.1M sodium phosphate buffer, (pH 7.2) for 1 minute, with continuous drops. The control cornea was then placed in a new petri dish, with fresh Life4° C. corneal preservation medium and microscopically evaluated for riboflavin diffusion through the epithelium and into the stroma using an Olympus IMT-2 fluorescent scope (485 nm/535 nm filter). Results of post-treatment analysis are shown in FIGS. 2A-2C (control sample at right).

The test cornea was treated with 1.0 ml of 0.1M sodium phosphate buffer, (pH 7.2) for 1 minute, with continuous drops; further treated with 0.5 mL of 0.1M sodium phosphate buffer, (pH 8.5) for 30 seconds with continuous drops; further treated with 1.0 ml of 10 mg/ml glutaric anhydride in 0.3M sodium phosphate buffer (pH 8.5) for 60 seconds with continuous drops (the buffered glutaric anhydride solution was mixed immediately prior to use); further treated with 0.5 mL of 0.1 M sodium phosphate buffer (pH 7.2) for 30 seconds, with continuous drops; further treated with 0.5 mL of riboflavin (1.46 mg/ml in PBS) for 60 seconds, with continuous drops; again further treated with 0.5 mL of riboflavin (1.46 mg/mL in PBS) for 60 seconds, with continuous drops; and rinsed with 1.0 ml of 0.1M sodium phosphate buffer, (pH 7.2) for 1 minute, with continuous drops. The test cornea sample was then placed in a new petri dish, with fresh Life4° C. corneal preservation medium and microscopically evaluated for riboflavin diffusion through the epithelium and into the stroma using an Olympus IMT-2 fluorescent scope (485 nm/535 nm filter). Results of post-treatment analysis are shown in FIGS. 2A-2C (test sample at left).

### Specular Microscopy Cell Analysis: Center Method

A single examiner performed all measurements. Corneal endothelium was evaluated utilizing a Konan Eyebank KeratoAnalyzer. The procedure for specular microscopy was as follows. One image from the central cornea was taken pre- and post-treatment. The centers of 100 contiguous cells were manually marked by the examiner for analysis by a built in validated software program. The computer automatically evaluated, calculated and displayed the following information as shown in FIG. 1C (test sample) and FIG. 2C (control sample):
1. Upper graph: Distribution of numbers of cell apices (unit: %).
2. Lower graph: Distribution of cell area (unit %).
3. Pachy: Corneal thickness.
4. AVE: average cell area (unit: μm2).
5. MAX: Maximum cell area (unit: μm2).
6. Minimum cell area (unit: μm2).
7. Number of cells analyzed.
8. CD: cell density (cells/mm2).
9. SD: Standard deviation of cell area.
10. CV: Coefficient of variation (SD/AVG×100).
11. 6A: Percent of hexagonal cells (hexagonality).

The coefficient of variation in cell size was used as the index of the extent of variation in cell area (polymegathism). Hexagonality was used as an index of variation in cell shape (pleomorphism).

### Results of Study

FIGS. 1A-1C show pre-treatment evaluation of human corneas 21-095656 OD (test sample, left) and 21-095656 OS (control sample, right). Both samples exhibited normal intact epithelium, stroma, and endothelium pre-treatment.

FIGS. 2A-2C shows post-treatment evaluation of the test and control samples, also demonstrating intact epithelium, stroma, and endothelium.

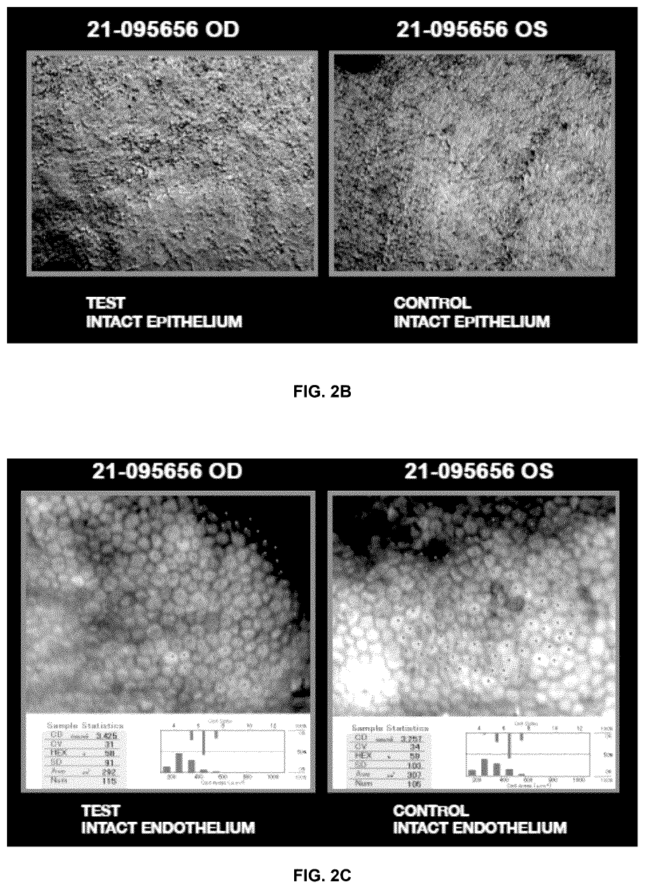

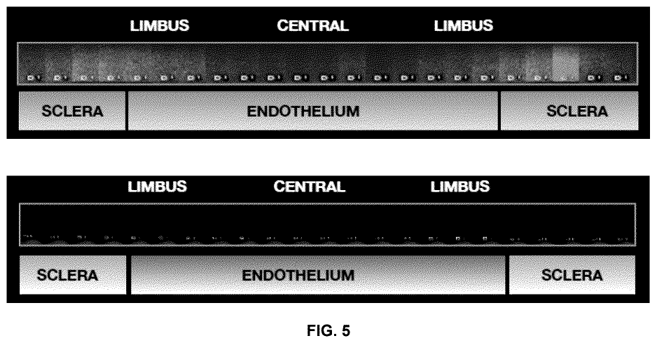

Test cornea 21-095656 OD exhibited riboflavin fluorescence within 2 minutes after the buffered glutaric anhydride solution treatment. The greatest riboflavin fluorescence was seen at the sclera endothelium interface. The riboflavin fluorescence exhibited a gradient effect as photographed from the sclera to central endothelium to sclera (FIG. 3 and top panel of FIG. 5). The riboflavin fluorescence was observed at the endothelial layer, indicating the riboflavin penetrated the epithelium and stroma. These results demonstrate the ability of glutaric anhydride to disrupt cell junctures to permit diffusion of riboflavin through the epithelium and deep into the corneal stroma.

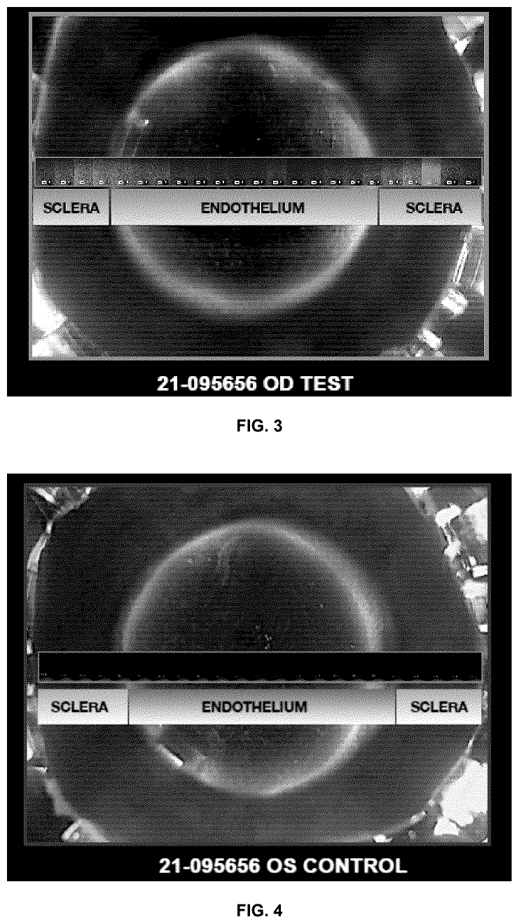

The control cornea 21-095656 OD exhibited no riboflavin fluorescence 2-10 minutes after the control treatment (FIG. 4 and bottom panel of FIG. 5). No riboflavin fluorescence was observed at the sclera-endothelial layer, indicating the riboflavin did not penetrate the epithelium and stroma. These results demonstrate that the cell junctions were still intact, preventing diffusion of riboflavin through the epithelium and stroma.

In conclusion, the buffered glutaric anhydride solution pre-treatment of the human cornea was an effective method of disrupting cell junctures to permit rapid diffusion of riboflavin through the cornea to the stroma. This buffered glutaric anhydride solution treatment did not adversely affect the corneal epithelium, stroma or endothelium as defined by the parameters of this study.

### Example 2

Another exemplary method according to the present disclosure is performed as follows. Drops of proparacaine HCl or a similar anesthetic are applied to the cornea for 1-2 minutes. The corneal surface is then exposed to 0.1-1.0 mL of a pretreatment buffer or solution at slightly alkaline pH ranging from 7.5-9.5, such as between 8.0 and 9.0, e.g., between 8.2-8.7. The buffer is sufficient to prevent the ultimate pH from dropping below 6.8. The buffer comprises sodium phosphate. Exposure time ranges from 15 seconds to 2 minutes, such as between 30 seconds and 1 minute.

Following exposure to the pretreatment buffer or solution, the corneal surface is exposed to an acylation agent. The cornea is first exposed to buffered glutaric anhydride (GA) solution or a similar anhydride, acid chloride, sulfonyl chloride, or sulfonic acid that is effective in disrupting epithelial cell junctures. The agent, e.g., GA, is dissolved in a suitable buffer at a concentration ranging from 1 mg/mL to 10 mg/mL, such as between 3 mg/mL and 5 mg/mL. The cornea is exposed to buffered GA solution for a period of time ranging from 15 seconds to 2 minutes, such as between 30 seconds and 1 minute. The corneal surface is then re-exposed to pretreatment buffer or solution for another 30 seconds to 1 minute.

Riboflavin (e.g., 0.5-5.0 mg/mL) is then applied to the cornea and subsequently diffuses into the stromal matrix. The eye is then exposed to UVA light at 370 nm, optionally under hyperoxic conditions.

While there is shown and described herein examples embodying aspects of the present disclosure, it is understood that various modifications and rearrangements of the methods may be made without departing from the spirit and scope of the underlying inventive concept and that the present disclosure is not limited to the examples herein shown and described.

1. A method of treating an eye of a subject, the method comprising:
administering an agent capable of disrupting corneal cell junctures to a corneal surface of the eye, the corneal surface comprising an intact epithelium and stroma; and
administering a therapeutically-effective amount of riboflavin to the corneal surface after or simultaneously with the agent;
wherein the riboflavin diffuses through the eye to penetrate the stroma within 10 minutes after administering the riboflavin.

2. The method of claim 1, wherein administering the agent to the corneal surface includes contacting the corneal surface with a buffered solution comprising the agent for at least 30 seconds.

3. The method of claim 2, wherein the solution is administered using an applicator placed against the corneal surface.

4. The method of claim 2, wherein the solution comprises the therapeutically-effective amount of riboflavin.

5. The method of claim 1, wherein the therapeutically-effective amount of riboflavin is administered within 5 minutes after administering the agent.

6. The method of claim 1, wherein the agent comprises an anhydride, an acid chloride, a sulfonyl chloride, or a sulfonic acid.

7. The method of claim 1, further comprising administering a buffer solution or physiological saline solution to the corneal surface before administering the agent to deprotonate free amines on corneal proteins.

8. The method of claim 7, wherein the buffer solution or physiological saline solution has a concentration ranging from 0.05 M to 1.0 M.

9. The method of claim 2, wherein the solution has a pH of from 7.5-9.0.

10. The method of claim 2, wherein the solution comprises dibasic sodium phosphate, monobasic sodium phosphate, disodium phosphate, or a mixture thereof.

11. The method of claim 2, wherein the solution has a concentration of the agent ranging from about 1.0 mg/mL to about 5.0 mg/mL.

12. The method of claim 1, wherein the agent dissociates molecular bridges between stromal collagen fibers.

13. The method of claim 1, wherein the therapeutically-effective amount of riboflavin is administered as a solution having a concentration of about 0.5 mg/mL to about 5.0 mg/mL riboflavin, the solution comprising riboflavin being the same or different than the solution comprising the agent.

14. The method of claim 1, wherein the therapeutically-effective amount of riboflavin is administered to the corneal surface by a plurality of drops.

15. The method of claim 1, wherein the therapeutically-effective amount of riboflavin corresponds to a total volume of riboflavin in solution ranging from about 0.1 mL to about 1.0 mL.

16. The method of claim 1, wherein at least 50% of the therapeutically-effective amount of riboflavin administered to the corneal surface diffuses into the stroma within 5 minutes of administering the riboflavin.

17. The method of claim 1, wherein at least 75% of the therapeutically-effective amount of riboflavin administered to the corneal surface diffuses into the stroma within 5 minutes of administering the therapeutically-effective amount of riboflavin.

18. The method of claim 1, wherein at least 90% of the therapeutically-effective amount of riboflavin administered to the corneal surface diffuses into the stroma within 5 minutes of administering the therapeutically-effective amount of riboflavin.

19. The method of claim 1, wherein at least 75% of the therapeutically-effective amount of riboflavin administered to the corneal surface diffuses into the stroma within 2 minutes of administering the therapeutically-effective amount of riboflavin.

20. The method of claim 1, further comprising irradiating the eye with UVA light.
